(19) United States
(12) Patent Application Publication
(10) Pub. No.: US 2014/0194857 A1
(43) Pub. Date: Jul. 10, 2014

(54) CATHETER WITH AN ENHANCED PUSHABILITY

(71) Applicants: ERAN EILAT, Herzilya (IL); CATHAWAY MEDICAL (2012) LTD., Raanana (IL); DR. EYAL BRESSLER LTD., Ramat Gan (IL)

(72) Inventor: Eran Eilat, Herzliya (IL)

(73) Assignees: ERAN EILAT, Herzliya (IL); CATHAWAY MEDICAL (2012) LTD., Raanana (IL); DR. EYAL BRESSLER LTD., Ramat Gan (IL)

(21) Appl. No.: 14/126,978

(22) PCT Filed: Jun. 21, 2012
(86) PCT No.: PCT/IL12/00244

§ 371 Date: Jan. 29, 2014

Related U.S. Application Data
(60) Provisional application No. 61/499,194, filed on Jun. 21, 2011.

(51) Int. Cl. A61M 25/00

(52) U.S. Cl. CPC A61M 25/0017
USPC 604/544

(57) ABSTRACT

A urinary catheter of enhanced pushability, for indwelling introduction into a patient's urethra having an effecter within the longitudinal bore of the catheter near its distal end to facilitate an easy introduction of the catheter into the body.

### FIELD OF THE INVENTION

A urinary catheter, such as a Foley catheter, for indwelling introduction into a patient's urethra having an effecter within the longitudinal bore of the catheter near its distal end to facilitate introduction of the catheter into the body. U.S. patent application Ser. No. 61/392,978 is incorporated in its eternity herein as a reference.

### BACKGROUND OF THE INVENTION

A catheter is a tube that can be inserted into a body cavity, duct, or vessel. Catheters thereby allow drainage, administration of fluids or gases, or access by surgical instruments.

There are several different types of catheters including, but not limited to: angiographic catheter- one through which a contrast medium is injected for visualization of the vascular system of an organ. Such catheters may have preformed ends to facilitate selective locating (as in a renal or coronary vessel) from a remote entry site. They may be named according to the site of entry and destination, such as femoral-renal and brachial-coronary. Arterial catheter- one inserted into an artery and utilized as part of a catheter-transducer-monitor system to continuously observe the blood pressure of critically ill patients. An arterial catheter also may be inserted for x-ray studies of the arterial system and for delivery of chemotherapeutic agents directly into the arterial supply of malignant tumors. Butterfly catheter- a metal needle with flexible plastic ‘wings’ and a short length of tubing. The ‘wings’ assist in placement and facilitate fixation with tape. Cardiac catheter- a long, fine catheter especially designed for passage, usually through a peripheral blood vessel, into the chambers of the heart under fluoroscopic control. See also cardiac catheterization. Cardiac biopsy catheter- introduced intravenously under the direction of fluoroscopy, can be positioned in the right or left ventricle and an endocardial biopsy obtained. Central venous catheter- a long, fine catheter inserted into a vein for the purpose of administering through a large blood vessel parenteral fluids (as in parenteral nutrition), antibiotics and other therapeutic agents. This type of catheter is also used in the measurement of central venous pressure. See also central venous catheterization.

column disk catheter- an indwelling device for continuous peritoneal dialysis. It is implanted within the peritoneal cavity, resting against the body wall. The attached Silastic tubing is used for infusing and draining the dialysate at intervals. Double-lumen catheter- one having two channels; one for injection and one for removal of fluid. Catheter drainage- a catheter left in place to keep the bladder drained. Preferably should have a one-way valve to avoid aspiration of air and infection. Elbowed catheter- a catheter bent at an angle near the beak. Indwelling catheter- one especially designed so that it is held in place in the urethra for the purpose of draining urine from the bladder. Over-the-needle catheter- a large-bore sharp needle housed with an indwelling stilette, inside a thin-walled plastic tube. An incision is made over the filled vein, the needle-cannula inserted, the stilette withdrawn, then the needle, leaving the plastic cannula in situ. Tracheal catheter- one with small holes at the terminal 1 inch, especially designed for removal of secretions during tracheal suctioning.

A urinary catheter is an elongated flexible tube usually made of natural rubber or silicone, such tube having a tip which is inserted into the urethra via the urethral meatus. The presently used ordinary urinary catheters are advanced through the urethra into a patient's bladder solely via axial loading. As stated in US 2011040290, there are few different types of urinary catheters: A Foley catheter, the most common, is provided at its tip with an inflatable balloon which secures the catheter in position within the bladder, not allowing its expulsion from the bladder. The balloon at the tip is inflated with sterile water or normal saline. Foley catheters are commonly made of silicone or rubber. A Robinson catheter is used for short term drainage of urine. Unlike the Foley catheter, it has no balloon on its tip and therefore cannot stay in place. A Coude catheter is generally more rigid than a Foley catheter. It has a curved tip, the purpose of which is to facilitate its insertion through urethral canal strictures such as in the case of benign prostatic hypertrophy. A Coude catheter may be provided with a balloon or not. A three ways irrigation catheter has a separate lumen to carry irrigation fluid into the bladder. It is commonly used to irrigate the bladder in case of hematuria with or without presence of clots within the bladder. The most common urinary catheter diameter sizes are 10 F (3.3 mm) to 28 F (9.3 mm). The length of urinary catheters varies, although they are usually approximately 40 cm long.

Common indications for placing a urinary catheter in a patient include: (i) acute or chronic urinary retention, both mechanical such as in the case of benign prostatic hypertrophy or non-mechanical such as in spastic bladder neck; (ii) the need to measure the urine output in critical care patients; (iii) incontinence; and (iv) patients post bladder or gynecological surgery.

Catheters are inserted into the patient via the urethral meatus. A gentle force is then applied to the proximal end of the catheter so that the catheter passes through the urethra and its distal end enters the bladder. After which, a balloon adjacent to the distal end of the catheter is inflated through an inflation lumen to retain the catheter in the patient. The proximal end of the catheter extends outside the patient's body and is connected to a drainage tube leading to a drainage bag. Urine passes through an opening or openings adjacent to the distal end of the catheter, through the drainage lumen extending through the catheter, and into the drainage tube to the bag for collection therein.

Current catheters have significant disadvantages. They might be difficult to insert because they lack column strength, partial obstruction of the pathway (mainly in man) or because of the flexibility needed to prevent discomfort to the patient during insertion and during use, especially for patients with indwelling catheters. The lack of column strength means that they can buckle or kink along their length during insertion or during use. They can also become twisted around their longitudinal axis during insertion mainly in the presence of partial obstruction along the pathway.

Buckling, kinking or twisting during insertion will make insertion significantly more difficult and can prevent insertion entirely. Such buckles, kinks and twists can occlude or partly occlude or narrow the catheter, limiting or preventing drainage of urine. Such buckles, kinks or twists can also very much increase the patient's discomfort. Patients trying to remove catheters to relieve their discomfort are a well-known problem.

well known problem of current catheters is the thickness of the walls needed to provide sufficient column strength to allow insertion. Thick walled catheters have diameters sufficiently large that significant discomfort is caused to the patient. Several attempts have been made to overcome this problem, such as U.S. Pat. No. 6,558,350B, which discloses a drainage catheter which comprises a woven mesh.

Current catheters are difficult to insert, can cause significant discomfort to the patient and frequently suffer from reduced flow of urine through them due to mechanical deformation during insertion or use. The present invention fulfils a long-felt need for a catheter that does not suffer from these defects.

### SUMMARY OF THE INVENTION

This present invention is discloses a flexible catheter of increased pushability, comprising a main longitudinal axis along which an encapsulated envelope with at least one open bore is in fluid communication between at least one proximal end and at least one distal end, wherein said catheter further comprises at least one effecter located within at least one portion of said bore, such that the stiffness of the catheter is increased along at least one portion of said main axis in at least one plane, whilst the flexibility of the catheter is not significantly reduced in other planes.

This present invention also discloses a flexible catheter of enhanced pushability, said catheter comprising a main longitudinal axis along which an envelope, having at least one open bore, is in fluid communication between the catheter's proximal and distal ends, wherein said catheter further comprises at least one effecter protruding within said bore and positioned along said envelope within at least one portion of the catheter, such that upon pushing the catheter towards the orifice of a body cavity, and advancing catheter's distal end throughout said cavity, the stiffness of the catheter is greater along said catheters' main axis than in other planes, whilst the flexibility of the catheter is not significantly reduced in other planes.

The present invention also discloses a flexible catheter of increased pushability as defined above, characterized by an elongated linear open bore encapsulated within a continuous envelope, said catheter having a main longitudinal axis, and at least one proximal end and at least one other distal end; wherein said catheter comprising at least one elongated effecter located within at least one portion of said bore and in parallel to said axis; said effecter provides said catheter with increased stiffness along said axis, whilst retaining the transverse and rotational flexibility of the catheter.

The present invention also discloses a flexible catheter of increased pushability as defined above, wherein said catheter is characterized by at least one fold along said longitudinal axis of said envelope, said fold is characterized by at least one inter-bore portion and at least one envelope-portion, wherein the outer surface of said catheter is continuous defining a well-encapsulated open bore.

The present invention also discloses a flexible catheter of increased pushability as defined above, structured as a Foley catheter, comprising a flexible envelope which defines an open-bore, and at least one effecter located (1) within said bore of said catheter, and (ii) in at least one portion of the horizontal plane of said bore, wherein said effecter provides said catheter with increased stiffness in said horizontal plane, whilst retaining the flexibility in the sagittal and coronal planes of said catheter.

The present invention also discloses a flexible catheter of increased pushability as defined above, wherein said envelope's outer surface defines a complete circle.

The present invention also discloses a flexible catheter of increased pushability as defined above, characterized by at least one fold along the longitudinal axis of said envelope, said fold is further characterized by at least one inter-bore portion and envelope-portion, wherein the outer surface of said catheter is continuous defining a well-encapsulated open-bore.

The present invention also discloses a flexible catheter of increased pushability as defined above, characterized by at least one fold along the longitudinal axis of said envelope, said fold is further characterized by at least one inter-bore portion and envelope-portion, wherein the outer surface of said catheter is continuous defining a well-encapsulated open-bore and wherein the base of the fold has sufficient flexibility that the sides of the fold may approach during insertion or during use, so that the effective diameter of the catheter may be decreased without significantly affecting the cross-sectional area of the open bore, so that fluid flow may be maintained in areas wherein the urethra is constricted.

The present invention also discloses a flexible catheter of increased pushability as defined above, wherein said effecter is selected from a group consisting of an effecter which has a triangular, flap-shaped, rectangular or polygonal cross-section; an effecter which has a curved, elliptical, oval or otherwise rounded cross-section; an effecter which has a leaf-like or fibrous-type or C-like or otherwise narrow cross-section; or any other irregular rounded shape; an effecter with a plurality of stems; an effecter where said stems join to form at least one joint cross-section; an effecter where at least one cross section divides or sub-divides into a plurality of members and cross-sections thereof; an effecter comprising at least one M-like, S-like, W-like, U-like, T-like, Y-like sub-structure; or any combination thereof.

The present invention also discloses a flexible catheter of increased pushability as defined above, wherein at least one portion of said effecter is made of a biocompatible polymeric material.

The present invention also discloses a flexible catheter of increased pushability as defined above, wherein is made of at least two biocompatible polymeric material with one being harder than the other.

The present invention also discloses a flexible catheter of increased pushability as defined above, wherein the effecter has flexible characteristics.

The present invention also discloses a flexible catheter of increased pushability as defined above, wherein the polymeric material is comprised of a silicone elastomer, rubber, latex or any mixture thereof.

The present invention also discloses a flexible catheter of increased pushability as defined above, wherein said effecter is affixed around the main axis such that a coil-like arrangement is provided.

The present invention also discloses a flexible catheter of increased pushability as defined above, wherein the effecter defines a pathway for infusing fluids via the proximal end to the distal end or vice versa.

The present invention also discloses a flexible catheter of increased pushability as defined above, wherein the catheter structure includes an intermediate region defined between the proximal region and the distal region.

The present invention also discloses a flexible catheter of increased pushability as defined above, wherein the catheter structure includes an infusion port located in the linear tubular structure.

The present invention also discloses a flexible catheter of increased pushability as defined above, wherein said effecter is hollow and the lumen thus formed is in fluid communication, in whole or in part, with the balloon retention mechanism of said catheter.

The present invention also discloses a flexible catheter of increased pushability as defined above, wherein said effecter is hollow and the lumen thus formed has fluid communication with an external port whereby it may be filled with saline or some other fluid or pressurized with air or some other gas, but said lumen has no fluid communication with the balloon retention mechanism of said catheter.

The present invention also discloses a flexible catheter of increased pushability as defined above, wherein said effecter is hollow and the lumen thus formed has no fluid communication with any external port and the said lumen is filled with saline or some other fluid or is pressurized with air or some other gas or contains air or some other gas at atmospheric pressure or contains air or some other gas at sub-atmospheric pressure.

The present invention also discloses a flexible catheter of increased pushability as defined above, wherein said effecter is hollow and is subdivided into two or more parts forming two or more lumens disposed either angularly about the main longitudinal axis, radially about such axis, or in any combination of these, wherein one or more of said lumens may be in fluid communication, in whole or in part, with the balloon retention mechanism of said catheter; one or more of said lumens may be in fluid communication with one or more external ports lumens may or may not share external ports; or one or more of said lumens may have no fluid communication with any external ports and be filled with saline or some other fluid or air or some other gas as a pressure which may be below atmospheric pressure, at atmospheric pressure, or above atmospheric pressure or any combination of these.

The present invention also discloses a method of using a Foley catheter which comprises at least one effecter. The method comprises steps of introducing a Foley catheter of increased pushability and non-kinking properties into the bladder of a patient and using the Foley catheter to drain urine from the bladder.

### BRIEF DESCRIPTION OF THE DRAWINGS

Further features of the invention, its nature and various advantages will be more apparent from the accompanying drawings and the following detailed description of the preferred embodiments, in which:

FIG. 1 presents a schematic view of the Foley catheter in accordance with one embodiment of the present invention;

FIG. 2 presents a coronal view of the effecter adjacent to the distal end of the tubular structure in accordance with one embodiment of the present invention;

FIG. 3 presents a coronal cross-section of the distal segment 16 in accordance with one embodiment of the present invention;

FIG. 4a-4b presents coronal cross sections of the catheter in accordance with two embodiments of the present invention.

FIG. 5 is a perspective view of the catheter in accordance with one embodiment of the present invention;

FIG. 6a-6p present coronal cross-sections of the prior art and various embodiments of the catheter which comprises one or more pushability enhancing effecters in accordance with the present invention;

FIG. 7 is a perspective view of a catheter comprises one or more spirally wounded pushability enhancing effecters in accordance with one embodiment of the present invention; and

FIGS. 8a and 8b are illustrations of a rotating extruding device for producing a catheter which comprises one or more spirally wounded pushability enhancing effecters in accordance with one embodiment of the present invention.

### DETAILED DESCRIPTION OF PREFERRED EMBODIMENTS

The present invention discloses a catheter with an enhanced pushability. The enhanced pushability is effected by means of an effecter, a structure of a material and of a shape that increase the stiffness of the catheter in the region of the said effecter without significantly affecting the stiffness in other directions. Said effecter extends inward into the bore of the catheter from the envelope or wall of the catheter.

A Foley catheter will be further presented herein, in a non-limiting manner, as a best mode.

Other catheters characterized by an enhanced pushability are possible and lie within the scope of the present invention.

FIG. 1 presents, in an out-of-scale manner, a Foley catheter generally designated with the reference numeral 10. The catheter 10 includes a catheter body 12 with a proximal end 14 and a distal end 16. The catheter also includes an inflation port 20 and a drainage port 22. The section 18 is double-walled. The gap between the two walls is connected via an inflation lumen to the inflation port 20. The outer wall of section 18 is thin to facilitate inflation of the outer wall (the ‘balloon’) so that the catheter may be retained in place in the bladder. The catheter may also include an infusion lumen (not shown) to permit introduction of fluid into the bladder, for example, to introduce antibiotics to cure bladder infections.

The catheter drainage lumen 22 extends from the proximal end 14 to the distal end 16. The distal end 16 includes one or more openings 26 in fluid communication with the drainage lumen 22 to facilitate drainage of urine from the bladder of a patient.

The effecter 40 (FIG. 3) attaches to the distal end of the Foley catheter body 12, extending from the catheter body 12 towards the distal end 16.

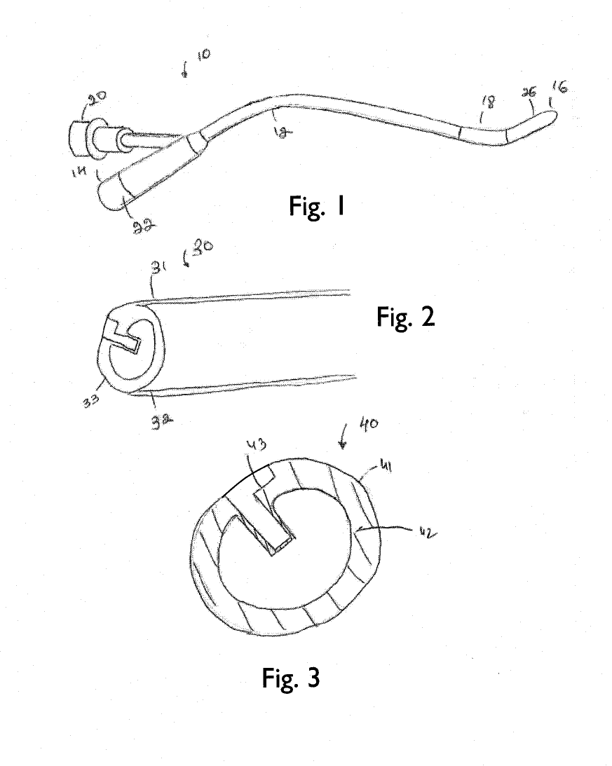

FIG. 2 presents, in an out-of-scale manner, the Foley catheter with effecter 33 attached, looking toward the distal end from the catheter body. in this embodiment, the catheter tube is a double-walled structure with an outer wall 31 of a material of a given stiffness and an inner wall 32 of a material of a different stiffness. By way of a non-limiting example, the outer wall may be comprised of a highly flexible biocompatible silicone elastomer whilst the inner wall may be comprised of a stiffer silicone elastomer.

The effecter of the present invention extends inward from the inner wall 32 of the catheter. In the present embodiment, it is continuous with said inner wall.

FIG. 3 presents, in an out-of-scale manner, a coronal cross-section of a catheter with an effecter that illustrates its structure, where said structure creates the improved pushability along the catheter body 12. The effecter is constructed of an outer wall 41, wherein the curved section comprises a closed tube, an inner wall 42 wherein the curved section comprises an open-sided tube and an effecter 43 wherein the effecter creates improved pushability of the catheter by increasing the stiffness of said catheter.

In this embodiment, the walls of the effecter are thin enough that a lumen is formed between the outer wall 41 and the effecter 43. This lumen may be continuous with the balloon lumen whereby the effecter is further stiffened when the balloon is inflated.

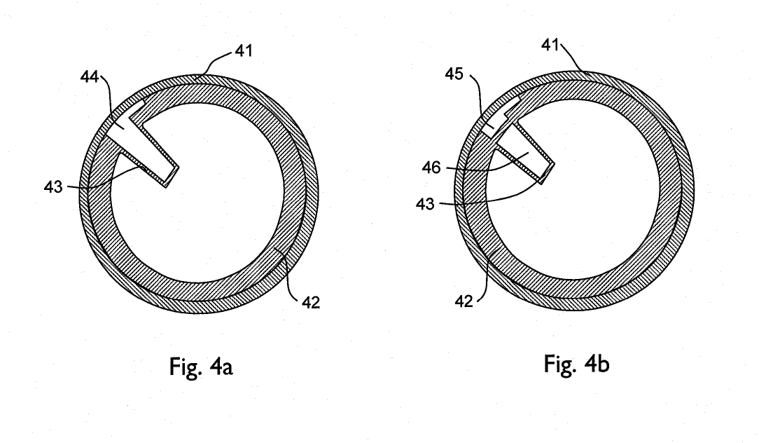

FIG. 4a-4b presents, in an out-of-scale manner, coronal cross-sections of two embodiments of the effecter wherein the effecter is thin walled so that there are one or more lumens between the effecter and the outer wall. In the embodiment of FIG. 4a the effecter lumen 44 is connected with the inflation port so that, when fluid passes through the inflation port, the thin outer wall 41 inflates to create a balloon which retains the catheter within the bladder, the fluid inside said lumen 44 stiffens the effecter. In the embodiment in FIG. 4b, outer effecter lumen 45 is connected with the inflation port whilst inner effecter lumen 46 is connected to a separate port so that said lumen 46 may be filled with air or some other gas under pressure or by saline or some other fluid and inflation of the balloon and stiffening of the effecter may be controlled independently.

Inflation of the effecter lumen or lumens may be used to increase the stiffness of the effecter. In both embodiments FIG. 4a and FIG. 4b, the effecter lumens 44 and 45 are filled at the same time as the balloon is expanded. Filling increases the stiffness of the effecter so that the distal end of the catheter does not bend or kink, ensuring that there is free passage for fluids from the bladder through the drainage lumen, into the drainage tube 22, and from thence into the drainage bag.

In the embodiment in FIG. 4b, the inner effecter lumen 46 is connected to a separate effecter port. The effecter lumen may be used to controllably increase the stiffness during insertion so that, for example, the stiffness may be controllably increased to gently straighten the catheter. The stiffness of the effecter lumen may also be increased in a controlled, repetitive manner, or pulsed, to gently ease the catheter past constrictions.

It is well known that the openings 26 that allow passage of urine from the bladder into the catheter are small and block easily. Pulsing of the pressure in the inner effecter lumen 46 could be used bend the distal end of the catheter slightly, both to clear small obstructions from said openings and, by slightly enlarging said openings, to allow said small obstructions to pass through said openings, from thence through the catheter into the drainage bag.

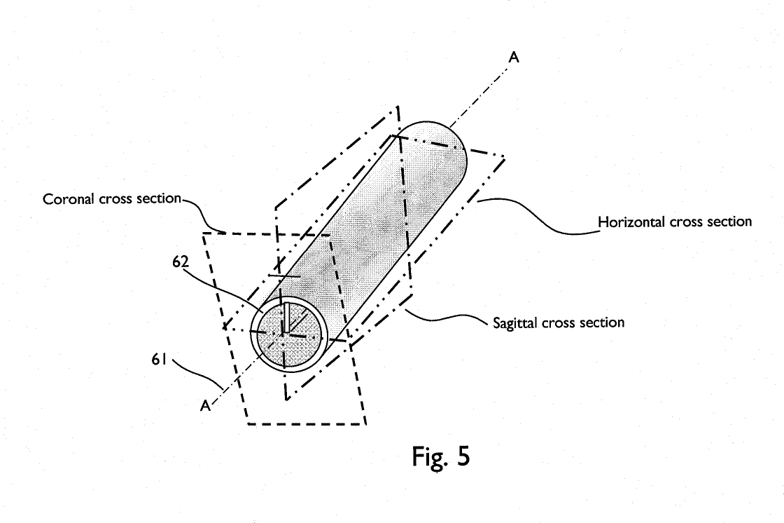

Reference is now made to FIG. 5, illustrating in an out-of-scale manner a perspective illustration of the catheter as defined above having various planes, such as a longitudinal axis, coronal plane, saggital plane and horizontal plane. The flexible catheter comprising a main longitudinal axis (A:A, 61) along which an encapsulated envelope 62 with at least one open bore is in fluid communication between at least one proximal end and at least one distal end, wherein said catheter further comprises at least one effecter 63 located within at least a portion of said bore, such that the stiffness of the catheter increases along at least a portion of said main axis, whilst the flexibility of the catheter is not significantly reduced in other planes.

It is in the scope of the invention, wherein the aforesaid flexible catheter is characterized by an elongated linear open-bore encapsulated within a continuous envelope 62. The catheter has a main longitudinal axis 61, and at least one proximal end and at least one other distal end. The catheter further comprises at least one elongated effecter 63 located within at least one portion of the bore and in parallel to the axis. The effecter provides the catheter with increased stiffness along axis A:A, whilst retaining the transverse and rotational flexibility of the catheter.

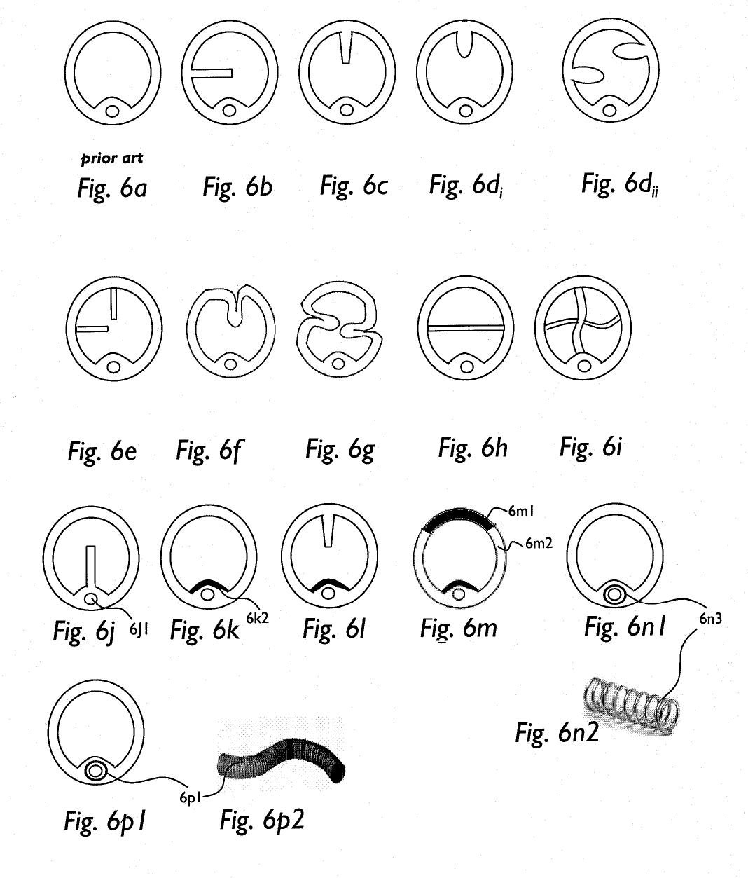

Reference is now made to FIGS. 6a to 6i. FIG. 6a illustrates, in an out-of-scale manner a coronal cross-section of a catheter of the prior art. FIGS. 6b to 6i illustrate, still in an out-of-scale manner, coronal cross-sections of various catheters being, in a non-limiting manner, a few embodiments of the invention. FIG. 6b shows a single rectangular effecter located in parallel to the horizontal cross-section. FIG. 6c shows a single triangular effecter located in parallel to the saggital cross-section. FIG. 6d, shows a single rounded-shape effecter located in parallel to the horizontal cross-section. FIG. 6d, shows a double parallel (here, e.g., an asymmetric arrangement) rounded-shape effecter located in parallel to the horizontal cross-section. FIG. 6e shows a two-rectangular effecters located in parallel to the horizontal and saggital cross-sections.

It is within the scope of the invention wherein the aforesaid planes are other than defined. Hence for example, one may locate the effecter(s) in any suitable location within the catheter's wall: at 12, 3, 6 or 9 o′clock; at 1, 4, 7, or 10 o′clock; at 2, 5, 8, and 11 o′clock etc.

According to one embodiment of the invention, at least one first effecter is located in one plane, for example at 12 o′clock along one segment (e.g., proximal side) of the catheter's shaft; and at least one second effecter is located in another plane, for example at 3 o′clock along another segment of the catheter's shaft (e.g., median portion or distal side).

According to another embodiment of the invention, at least one effecter is made of a first material (e.g. relatively flexible rubber) and (i) at least one second effecter or (ii) a portion of said first effecter, is made of a second material (e.g., relatively inflexible rubber).

Reference is now made to FIG. 6f and FIG. 6g, presenting flexible catheters that are characterized by one fold and two folds, respectively, along said longitudinal axis of the envelope. The fold of FIG. 6f is parallel to the saggital cross-section whereas the two folds of FIG. 6g are parallel to the horizontal cross-section. Those folds are characterized by at least one inter-bore portion and envelope-portion, wherein the outer envelopes of the catheters are continuous defining a well-encapsulated open-bore.

Reference is now made to FIG. 6h and FIG. 6i, presenting flexible catheters having (i) a single side-to side rectangular effecter, here for example located in parallel to the horizontal cross section, and (ii) two side-to-side curved effecters, here for example located in parallel to both horizontal and saggital cross sections, respectively.

Reference is now made to FIG. 6i to FIG. 6p, presenting a cross section of flexible catheters having a single side-to side rectangular effecter located or in connection with the inner envelope. FIG. 6i presents a cross section the catheter where the effecter is connected with the inner envelope 6j1. FIG. 6k presents a cross section the catheter where the effecter is at least one portion of envelope 6k2. This portion is made from polymers being more rigid and less flexible than the outer envelope and the other portions of the inner envelope. FIG. 61 presents a cross section the catheter where the effecter is not connected with the inner envelope and at least one portion of inner envelope is made by less flexible polymeric compositions. FIG. 6m presents a cross section the catheter where the effecter is at least one portion of the outer envelope (e.g., 6m 1) which is made by less flexible polymeric compositions and at least one portion of inner envelope which is made less flexible polymeric compositions. FIG. 6n 1 presents a cross section the catheter where the effecter is a spring-like effecter 6n3 (e.g., a metallic spring like or coil-like member, a Nitinol™-made spring-like member or a polymeric spring-like member, See FIG. 6n2) which is located within the inner envelope. FIG. 6p1 presents a cross section the catheter where the effecter is a tube-like or pipe-like effecter or accordion-like open tube 6p1 (See FIG. 6p2) which at least partially located within the inner envelope.

It is further in the scope of the invention, wherein the aforesaid effecter is an elongated member being substantially parallel to the catheter's main longitudinal axis.

It is further in the scope of the invention, wherein the aforesaid effecter is an elongated member spirally winding in respect to the catheter's main longitudinal axis.

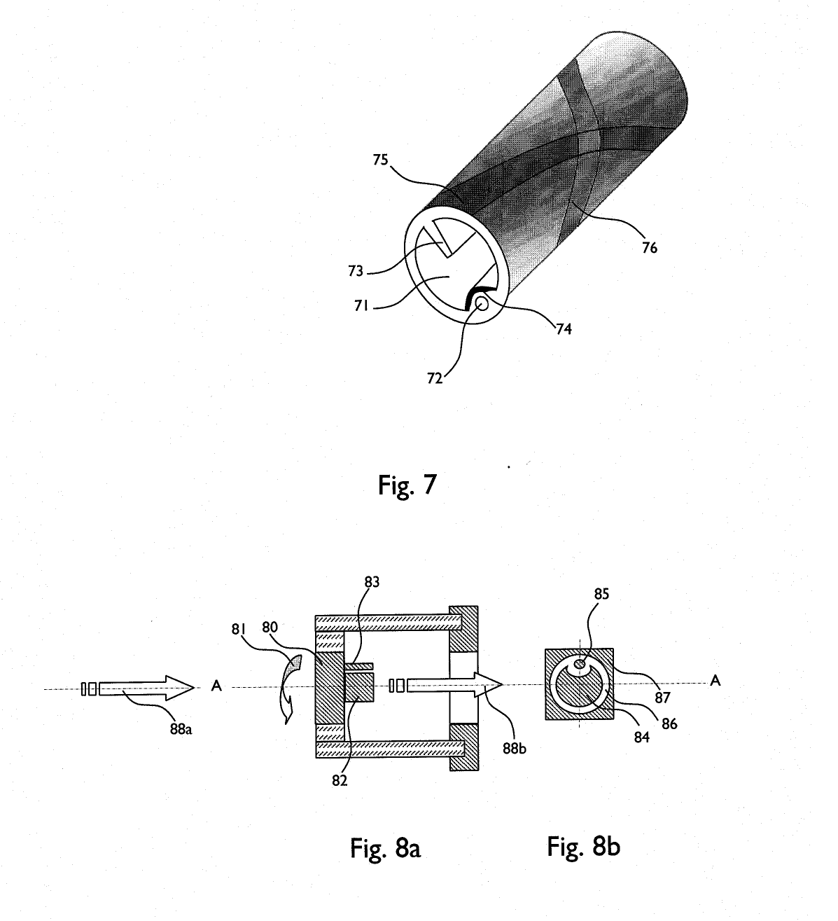

Reference is now made to FIG. 7 presenting in a non-limiting manner a schematic three-dimensional presentation of one of the catheter's segments. The catheter of this example comprises a main respectively large bore 71 and a secondary smaller bore 72. One pushability-enhancing effecter 73 is located within the main. The secondary bore is at least partially enveloped by material 74 being more rigid and less flexible material, comparing other portions of the envelope. The said effecter 73 and the said rigid portion 74 is spirally wounded in respect to the catheter's main longitudinal axis, see marks 75 and 76, respectively.

It is further in the scope of the invention, wherein the aforesaid flexible catheter defined above is structured as a Foley catheter. This new Foley catheter of enhanced pushability and kink-resistance comprises a flexible envelope which defines an open-bore, and at least one effecter located (i) within said bore of said catheter, and (ii) in at least one portion of the horizontal plane of said bore, wherein the effecter provides the catheter with increased stiffness in said horizontal plane, whilst retaining the flexibility in the sagittal and coronal planes of said catheter.

It is further in the scope of the invention, wherein the aforesaid flexible catheter of increased pushability, e.g., the Foley catheter is characterized by an envelope's outer diameter which defines a complete circle.

It is further in the scope of the invention, wherein the aforesaid flexible catheter of increased pushability, e.g., the Foley catheter is characterized by at least one fold along the longitudinal axis of said envelope, said fold is further characterized by at least one inter-bore portion and envelope-portion, wherein the outer envelope of said catheter is continuous defining a well-encapsulated open-bore.

It is further in the scope of the invention, wherein effecter has, for example, and in a non-limiting manner a shape that is selected from one or more member of the following group: a triangular, flap-shaped, rectangular or polygonal cross-section; an effecter which has a curved, elliptical, oval or otherwise rounded cross-section; an effecter which has a leaf-like or fibrous-type or C-like or otherwise narrow cross-section; an effecter with a plurality of stems; an effecter where said stems join to form at least one joint cross-section; an effecter where a at least one cross section divides or sub-divides into a plurality of members and cross-sections thereof; an effecter comprising at least one M-like, S-like, W-like, U-like, T-like, Y-like sub-structure; or any combination thereof.

It is lastly in the scope of the invention to disclose a method of the production of either linear or spirally wounded pushability enhancing effecter. The method comprises, inter alia, steps of extruding silicone elastomer, rubber, latex or any mixture thereof throughout a static extruding pre-shaped nuzzle to continuously obtaining the catheter's outer and inner envelopes as defined above. Alternatively or additionally, the method comprises, inter alia, steps of extruding silicone elastomer, rubber, latex or any mixture thereof throughout a rotating extruding pre-shaped nuzzle to continuously obtaining the catheter's outer and inner envelopes as defined above.

Reference is thus made to FIG. 8a and FIG. 8b, schematically presenting in an out-of-scale manner a rotating extruding's pre-shaped nuzzle is a side and front view, respectively. This rotating mechanism accept inlet melt (88a) while nuzzle (80) is rotating (81). The mechanism comprises main aperture (82) permitting the flow or at least one first melt forming the envelopes of catheter. This first melt is made of respectively flexible and non-rigid materials. Whilst rotating, a secondary aperture (83) is rotating, permitting a batch-wise or continuous flow or at least one second melt forming at least one portion of the envelopes of catheter and/or pushability enhancing effecters as defied in any of the above. This second melt is made of respectively non-flexible and more rigid materials.

1. A flexible catheter of enhanced pushability, said catheter comprising a main longitudinal axis along which an envelope, having at least one open bore, is in fluid communication between the catheter's proximal and distal ends, wherein said catheter further comprises at least one effecter protruding within said bore and positioned along said envelope within at least one portion of the catheter, such that upon pushing the catheter towards the orifice of a body cavity, and advancing catheter's distal end throughout said cavity, the stiffness of the catheter is greater along said catheters' main axis than in other planes, whilst the flexibility of the catheter is not significantly reduced in other planes.

2. A flexible catheter of enhanced pushability according to claim 1, characterized by an elongated linear open-bore encapsulated within a continuous envelope, said catheter having a main longitudinal axis, and at least one proximal end and at least one other distal end; wherein said catheter comprising at least one elongated effecter located within at least one portion of said bore and in parallel to said axis; said effecter provides said catheter with increased stiffness along said axis, whilst retaining the transverse and rotational flexibility of the catheter.

3. A flexible catheter of enhanced pushability of claim 1, wherein said catheter is characterized by at least one fold along said longitudinal axis of said envelope, said fold is characterized by at least one inter-bore portion and at least one envelope-portion, wherein the outer envelope of said catheter is continuous defining a well-encapsulated open-bore.

4. A flexible catheter of enhanced pushability as defined above, characterized by at least one fold along the longitudinal axis of said envelope, said fold is further characterized by at least one inter-bore portion and envelope-portion, wherein the outer surface of said catheter is continuous defining a well-encapsulated open-bore and wherein the base of the fold has sufficient flexibility that the sides of the fold may approach during insertion or during use, so that the effective diameter of the catheter may be decreased without significantly affecting the cross-sectional area of the open bore, so that fluid flow may be maintained in areas wherein the urethra is constricted.

5. A flexible catheter of enhanced pushability according to claim 1, structured as a Foley catheter, comprising a flexible envelope which defined an open-bore, and at least one effecter located (i) within said bore of said catheter, and (ii) in at least one portion of the horizontal plane of said bore, wherein said effecter provides said catheter with increased stiffness in said horizontal plane, whilst retaining the flexibility in the sagittal and coronal planes of said catheter.

6. A flexible catheter of enhanced pushability of claim 1, wherein said envelope's outer surface defines a complete circle.

7. A flexible catheter of enhanced pushability of claim 1, characterized by at least one fold along the longitudinal axis of said envelope, said fold is further characterized by at least one inter-bore portion and envelope-portion, wherein the outer envelope of said catheter is continuous defining a well-encapsulated open-bore.

8. The catheter of claim 1, wherein said effecter is selected from a group consisting of an effecter which has a triangular, flap-shaped, rectangular or polygonal cross-section; an effecter which has a curved, elliptical, oval or otherwise rounded cross-section; an effecter which has a leaf-like or fibrous-type or C-like or otherwise narrow cross-section; an effecter with a plurality of stems; an effecter where said stems join to form at least one joint cross-section; an effecter where a single cross section divides or sub-divides into a plurality of members and cross-sections thereof; an effecter comprising at least one M-like, S-like, W-like, U-like, T-like, Y-like sub-structure; or any combination thereof.

9. The catheter of claim 1, wherein at least one portion of said effecter is made of a biocompatible polymeric material.

10. The catheter of claim 1, wherein the effecter has flexible characteristics.

11. The catheter of claim 1, wherein the polymeric material is comprised of a silicon elastomer.

12. The catheter of claim 1, wherein said effecter is affixed around the main axis such that a coil-like arrangement is provided.

13. The Foley catheter of claim 1, wherein the effecter defines a pathway for infusing fluids via the proximal end to the distal end.

14. The Foley catheter of claim 1, wherein the catheter structure includes an intermediate region defined between the proximal region and the distal region.

15. The Foley catheter of claim 1, wherein the catheter structure includes an infusion port located in the linear tubular structure.

16. The Foley catheter of claim 1, wherein said effecter is hollow and the lumen thus formed is in fluid communication, in whole or in part, with the balloon retention mechanism of said catheter.

17. The Foley catheter of claim 1, wherein said effecter is hollow and the lumen thus formed has fluid communication with an external port whereby it may be filled with saline or some other fluid or pressurized with air or some other gas, but said lumen has no fluid communication with the balloon retention mechanism of said catheter.

18. The Foley catheter of claim 1, wherein said effecter is hollow and the lumen thus formed has no fluid communication with any external port and the said lumen is filled with saline or some other fluid or is pressurized with air or some other gas or contains air or some other gas at atmospheric pressure or contains air or some other gas at sub-atmospheric pressure.

19. The Foley catheter of claim 1, wherein said effecter is hollow and is subdivided into two or more parts forming two or more lumens disposed either angularly about the main longitudinal axis, radially about such axis, or in any combination of these, wherein one or more of said lumens may be in fluid communication, in whole or in part, with the balloon retention mechanism of said catheter; one or more of said lumens may be in fluid communication with one or more external ports lumens may or may not share external ports; or one or more of said lumens may have no fluid communication with any external ports and be filled with saline or some other fluid or air or some other gas as a pressure which may be below atmospheric pressure, at atmospheric pressure, or above atmospheric pressure or any combination of these.
